(19) United States
(12) Patent Application Publication
(10) Pub. No.: US 2008/0220486 A1
(43) Pub. Date: Sep. 11, 2008

(54) Method for growing photosynthetic organisms

(75) Inventor: Herman Weiss, Rishon Lezion (IL)

Correspondence Address: NATH & ASSOCIATES, 112 South West Street, Alexandria, VA, 22314, US

(73) Assignee: SEAMBIOTIC LTD., Tel Aviv (IL)

(21) Appl. No.: 12/073,495

(22) Filed: Mar. 6, 2008

Related U.S. Application Data
(60) Provisional application No. 60/905,605, filed on Mar. 8, 2007.

(51) Int. Cl. C12P 7/64; C12P 1/00; C12P 7/06; C12P 5/02; C12N 1/12

(52) U.S. Cl. 435/134; 435/257.1; 435/41; 435/161; 435/167

(57) ABSTRACT

A method of growing photosynthetic organisms comprising providing the organisms with flue gases from a fossil-fuel power plant, the gases being previously treated by desulfurization. The carbon dioxide (CO₂) concentration of the flue gases may be increased over the CO₂ concentration as released from the power plant. Also disclosed is a method for producing ω fatty acids and bio-fuels comprising growing microalgae by providing said microalgae with flue gases from a fossil-fuel power plant.

### FIELD OF THE INVENTION

This invention relates to bioconversion by photosynthetic organisms of CO₂ in flue gases from a power station.

### BACKGROUND OF THE INVENTION

One of the greatest current environmental concerns both for the near term as well as for the future is the dramatic increase in airborne greenhouse gases, particularly carbon dioxide (CO₂). Atmospheric CO₂ concentration has been increasing steadily since the industrial revolution. It has been widely accepted that while the atmospheric CO₂ concentration was about 280 ppm before the industrial revolution, it has increased to 315 ppm in 1959 and to 370 ppm in 2001. The rising CO₂ concentration has been reported to account for half of the greenhouse effect that causes global warming. Although the anthropogenic CO₂ emissions are small compared to the amount of CO₂ exchanged in the natural cycles, the discrepancy between the long life of CO₂ in the atmosphere (50-200 years) and the slow rate of natural CO₂ sequestration processes leads to a CO₂ build up in the atmosphere. The IPCC (Intergovernmental Panel on Climate Change) opines that “the balance of evidence suggests a discernible human influence on the global climate”. Therefore, it is necessary to develop cost effective CO₂ management schemes to curb its emission.

The major contributors of these gases are the exhaust of motor-driven vehicles and the flue gas of fossil-fuel fired power plants. Intensive research has been invested during the last two decades in finding ways of reducing the amount of CO₂ in the gases emitted to the atmosphere. Many of the envisaged CO₂ management schemes consist of three parts—separation, transportation and sequestration of CO₂. The cost of separation and compression of CO₂ (for transportation of CO₂ in liquid state) is estimated at $30-50 per ton CO₂, and transportation and sequestration would cost about $25 per ton of CO₂. The dominating costs associated with the current CO₂ separation technologies necessitate development of economical alternatives.

Historically, CO₂ separation was motivated by enhanced oil recovery. Currently, industrial processes such as limestone calcinations, synthesis of ammonia and hydrogen production require CO₂ separation. Absorption processes employ physical and chemical solvents such as Selexol and Rectisol, MEA and KS-2. Adsorption systems capture CO₂ on a bed of adsorbent materials. CO₂ can also be separated from the other gases by condensing it out at cryogenic temperatures. Polymers, metals such as palladium, and molecular sieves are being evaluated for membrane based separation processes.

Concern over the increased concentration of CO₂ in the atmosphere and its effect on global climate change has increased the awareness and investigations for reducing CO₂ emissions. Most of the methods for CO₂ mitigation require CO₂ in a concentrated form, while the CO₂ emitted from coal-fired power plants is mixed with N₂, water vapor, oxygen, and other impurities, and is present at a low ˜12-15% concentration. Therefore, capturing CO₂ from flue gas in a concentrated form is a critical step that precedes a variety of proposed sequestration approaches.

One of the most discussed ways for the sequestration of CO₂ from power plant flue gases is the bioconversion of CO₂ and solar energy to biomass by photosynthesis. Bioconversion of the power station's CO₂ emissions can be especially efficient in countries with high solar activity, such as in Mediterranean countries. In Western Europe, there are examples showing that when flue gases are supplied by natural gas-fired power stations to greenhouses, the CO₂ emissions are converted from a problematic source of climate change into a positive factor for agriculture. Fossil-fuel-burning power stations are often situated near seashores or estuaries. It is known that photosynthesis is much more efficient in algae than in terrestrial plants, conversion of solar energy reaching 9-10%. Microalgae have been used to fix CO₂ from the flue gas emitted by coal-fired thermal power plants. A Chlorella species was found to grow under such conditions (Maeda, K; Owada, M; Kimura, N; Omata, K; Karube, I, CO₂ fixation from the flue gas on coal-fired thermal power plant by microalgae, Proceedings of the 2^{nd} Intl. Confer. Carbon Dioxide Removal, 1995, Energy Conversion and Management, V. 36, no. 6-9, p. 717-720).

U.S. Pat. Nos. 4,398,926, 4,595,405, 4,681,612 and 7,153,344 disclose methods for removal of impurities from a gas.

WO 2007/011343 discloses a photobioreactor apparatus containing a liquid medium comprising at least one species of photosynthetic organism. The apparatus may be used as part of a fuel generation system or in a gas treatment process to remove undesirable pollutants from a gas stream.

Biomass in the form of agricultural crops, agricultural and forestry residues (captive and collected), energy crops (grasses, algae, and trees) and animal wastes can be converted by thermo-chemical pretreatment, enzymatic hydrolysis, fermentation, combustion/co-firing, gasification/catalysis, gasification/fermentation or by pyrolysis, to fuels—bioethanol/biodiesel/biogas, power—electricity and heat, and chemicals—organic acids, phenolics/solvents, chemical intermediates, plastics, paints and dyes.

Omega-3 fatty acids and their counterparts, n-6 fatty acids, are essential polyunsaturated fatty acids (PUFA) because they cannot be synthesized de novo in the body. The major sources of 18-carbon n-3 essential fatty acids (linolenic acid [LNA]), are flax seed, soybean, canola, wheat germ, and walnuts oils. Linoleic acid (LA), the 18 carbon n-6 essential fatty acid, is found in safflower, corn, soybean, and cottonseed oils; meat products are a source of the LC n-6 fatty acid, arachidonic acid (AA) (C20:4n-6). The 20-and 22-carbon PUFA sources are fish and fish oils.

The 18-carbon PUFAs derived from plant sources can be converted (although not efficiently) to their longer chain and more metabolically active forms: AA, eicosapentaenoic acid (EPA) (C20:5n-3), and docosahexaenoic acid (DHA) (C22:6n-3). The conversion of n-3 and n-6 fatty acids uses the same enzyme pools. AA and EPA, both 20-carbon fatty acids, are precursors to various eicosanoids. Most research has focused on prostaglandins, thromboxanes, and leukotrienes derived from AA and EPA. AA is a prominent precursor to highly active eicosanoids, while EPA is a precursor to less metabolically active eicosanoids. AA and EPA reside in the membrane phospholipid bilayer of cells. AA is a precursor to series 2 prostaglandins and thromboxanes and series 4 leukotrienes. The series 2 and 4 eicosanoids metabolized from AA can promote inflammation, and also can act as vasoconstrictors, stimulate platelet aggregation and are potent chemotoxic agents dependent on where in the body the eicosanoids are activated. EPA is a precursor to series 3 prostaglandins and thromboxanes and series 5 leukotrienes; they are less potent than the series 2 and 4 counterparts and act as vasodilators and anti-aggregators. EPA is considered anti-inflammatory.

DHA is a 22-carbon fatty acid and therefore not directly converted to eicosanoids; however, DHA can be retro-converted to EPA. DHA is a prominent fatty acid in cell membranes, it is present in all tissues and is especially abundant in neural (60% of the human brain is comprised of PUFAs, predominately DHA) and retinal tissue and essential in visual and neurologic development.

### SUMMARY OF THE INVENTION

It is an object of the present invention to provide a method for growing photosynthetic organisms using flue gases from a fossil-fuel power plant.

In a first aspect of the invention, there is provided a method of growing photosynthetic organisms comprising providing said photosynthetic organisms with flue gases from a fossil-fuel power plant, the gases being treated by desulfurization.

In a preferred embodiment of this aspect of the invention, the carbon dioxide (CO₂) concentration of the flue gases is increased over the CO₂ concentration as released from the power plant.

In a second aspect of the invention, there is provided a method of growing photosynthetic organisms comprising providing said photosynthetic organisms with flue gases from a fossil-fuel power plant wherein the CO₂ concentration of said flue gases is increased over the CO₂ concentration as released from the power plant.

The fossil-fuel may be any type of fossil-fuel such as coal (e.g. lignite), petroleum (oil), natural gas, biomass, etc. Examples of petroleum include crude oil, light oil and heavy oil. In a preferred embodiment, the fossil fuel is coal. Non-limiting examples of types of coal which may be used in the methods of the invention include South African, TCOA; South African, KFT; South African, Amcoal; South African, Glencore; South African, Middleburg; Australian, Ensham; Australian, Saxonvale; Australian, MIM; Colombian, Carbocol; Colombian, Drummond; Indonesian, KPC; South African, Anglo; Consol, USA; and Australian, Warkworth.

The term “desulfurization” includes any method which removes sulfur dioxide (SO₂) from a mixture of gases. Desulfurization may at times be referred to as “flue gas desulfurization” (FGD), which is a variety of the current state-of-the art technologies used for removing SO₂ from the exhaust flue gases emitted from fossil-fuel power plants. Examples of FGD methods include: (1) wet scrubbing, using a slurry of sorbent, usually limestone or lime, to scrub the gases; (2) spray-dry scrubbing using similar sorbent slurries; and (3) dry sorbent injection systems. In a preferred embodiment, the FGD is by wet scrubbing.

Flue gas emitted from a fossil-fuel power plant (also called stack gas) is usually composed of CO₂ and water vapor as well as nitrogen and excess oxygen remaining from the intake combustion air. It also can contain a small percentage of pollutants such as particulate matter, carbon monoxide, nitrogen oxides, sulfur oxides, volatile organic compounds (VOC) and very small quantities of heavy metals in gaseous phase. The CO₂ concentration in coal burning flue gas is generally 12-16%. All percentages are Vol/Vol, unless otherwise indicated.

In accordance with the methods of the invention, the CO₂ concentration of flue gases is increased over the CO₂ concentration as released from the power plant. In one embodiment, the CO₂ concentration of flue gases is significantly increased over the CO₂ concentration as released from the power plant. The term “significantly increased” refers to an increase of at least 1.5 times (50%), preferably an increase of at least 2 times (100%), more preferably at least 3 or 4 times (200-300%), still more preferably at least 5 or 6 times (400-500%). Increased CO₂ concentration ranges may be 17-22%, 23-27%, 28-35%, or 36-50%. In each specific case, the advantage of increasing the CO₂ concentration must be balanced with its cost.

The CO₂ concentration of the flue gases may be increased (or separated) by any of the many conventional methods well known to the average skilled man of the art. In one embodiment, the separation is carried out using a membrane. U.S. Pat. No. 4,398,926 teaches the separation of hydrogen from a high-pressure stream, using a permeable membrane. U.S. Pat. No. 4,681,612 deals with the separation of landfill gas, and provides for the removal of impurities and carbon dioxide in a cryogenic column. Methane is then separated by a membrane process. The temperature of the membrane is 80° F. U.S. Pat. No. 4,595,405, again, combines a cryogenic separation unit and a membrane separation unit. The membrane unit is operated with gas at or near ambient temperature. The contents of all of the aforementioned patents are incorporated herein by reference.

In another embodiment, the CO₂ concentration is increased using a carbon molecular sieve membrane. The carbon molecular sieve membrane may be a hollow fibre type. An example of the use of such a molecular sieve membrane for CO₂ separation is disclosed in U.S. Pat. No. 7,153,344, whose entire contents are incorporated herein by reference. One example of using this separation method in one embodiment of the method of the invention is described in detail below.

In one embodiment of this aspect of the invention, the system for increasing the concentration of CO₂ includes a low pressure preliminary condensation tank to remove water from the FGD treated gas.

In another embodiment, the system includes—for the cases where membranes are applied—a tank (filter) with special activated carbon for reduction of sulfur and/or nitrogen oxides for membrane protection.

In a further embodiment, the system includes a compressor(s) station with one or more of control devices, valves, pipes, instruments and speed control facilities.

In a further embodiment, the system includes a high pressure condensation tank equipped with condensate collecting and evacuation facilities.

In a still further embodiment, the system includes a membrane unit including one or more of booster compressor(s), membrane module(s), control facilities and instruments.

In another embodiment, the system includes a gas receiver tank.

In another embodiment, the system includes aeration devices (also known as atomizers) such as porous aeration devices for dispersion of the carbon dioxide-rich gas in the microalgae ponds. Such devices are manufactured by the KREAL company.

In still another embodiment, the system includes a separate pipeline for supply of the above condensate to the algae farm and a system for its distribution among the ponds.

Two membrane operations which appear to have potential are gas separation and gas absorption. The CO₂ is removed by each process with the aid of gas separation membranes and gas absorption membranes (optionally in combination with monoethanolamine (MEA)). Examples of gas separation membranes which may be used are polyphenyleneoxide and polydimethylsiloxane. The former has good CO₂N₂ separation characteristics (with very low CO₂ content in the gas stream) and costs about 150 US$/m². The latter at 300 US$/m² is a good CO₂/O₂ separator. For the gas absorption membranes, porous polypropylene may be used.

The photosynthetic organisms used in the method of the invention are preferably microalgae. Microalgae are microscopic plants that typically grow suspended in water and carry out photosynthesis, thereby converting water, CO₂ and sunlight into O₂ and biomass. In an embodiment of the invention, the microalgae are marine microalgae, or phytoplankton, i.e. they grow in seawater or salt water. Examples of marine microalgae include diatoms (Bacillariophyta), the dinoflagellates (Dinophyta), the green algae (Chlorophyta) and the blue-green algae (Cyanophyta). Other microalgae include one or more of the species Phaeodactylum, Isochrysis, Monodus, Porphyridium, Spirulina, Chlorella, Botryococcus, Cyclotella, Nitzschia and Dunaliella. In another embodiment, the marine microalgae are from the Bacillariophyta class, and in a preferred embodiment, are from the Skeletonema order. In another embodiment, the marine microalgae are from the class Eustigmatophytes, and in a preferred embodiment, are from the Nannochloropsis sp. order. In a further embodiment, the marine microalgae are from the class Chlorophyta, and in a preferred embodiment, are from the Chlorococcum, Dunaliella, Nannochloris, and Tetraselmis species.

Marine microalgae are a source of ω (omega) 3 fatty acids. Microalgae contain a wide range of fatty acids in their lipids. Of particular importance is the presence of significant quantities of the essential polyunsaturated fatty acids (PUFA), ω6-linoleic acid (C18:2) and ω3-linolenic acid (C18:3), and the highly polyunsaturated ω3 fatty acids, octadecatetraenoic acid (C18:4), eicosapentaenoic acid (EPA, C20:5) and docosahexaenoic acid (DHA, C22:6). Microalgae can also serve as a source of biofuel such as biodiesel and bioethanol.

Thus additional aspects of the invention include:
A method for producing ω fatty acids comprising growing microalgae by providing said microalgae with flue gases from a fossil-fuel power plant, and separating the ω fatty acids from the microalgae.
A method for producing a biofuel, such as biodiesel and bioethanol comprising growing microalgae by providing said microalgae with flue gases from a fossil-fuel power plant, and separating the biofuel from the micro algae.

Still another aspect of the invention relates to a method of harvesting microalgae, and in particular Skeletonema, from a cultivation medium, wherein the microalgae are grown using flue gases from a fossil-fuel power plant. It has been discovered that such microalgae undergo auto-flocculation and sedimentation.

Cultivation of microalgae with intensive CO₂ enrichment by stack gases is an efficient way for both conversion of solar energy into useful biomass and mitigation of power stations carbon emissions. In order to increase the cultivation efficiency one has to provide maximal exposure of the algae to sunlight (done by mixing) and has to use the fossil fuel fired power stations fuel gases as the CO₂ source.

Mixing is achieved by wave generation in the ponds created by various wave makers.

Flue gases are a cheap and unlimited source of CO₂, but its low concentration and difficulty to be liquefied, limits their application. The disadvantage of their use as compared with pure CO₂ is the necessity to supply and to disperse large volumes of the gases; if the ponds are situated at a distance from the power station stack, the advantages of this cheap CO₂ source use should be reconsidered. This problem can be solved by application of the membrane technologies, enabling a considerable increase in the CO₂ concentration of the flue gas stream to the cultivation site. The efficient dispersion of the gases in the seawater ponds with low head losses can be realized by the application of diffusers.

A further aspect of the invention relates to a method of harvesting microalgae from a cultivation medium. The method comprises growing the microalgae using flue gases from a fossil-fuel power plant, the gases being separated by desulfurization, allowing the microalgae to precipitate and harvesting the precipitated microalgae.

In a preferred embodiment, the microalgae are Skeletonema.

In a still further aspect of the invention, there is provided a method of removing protozoan contaminants from an aqueous medium comprising microalgae, the medium having a first pH value. The method comprises lowering the pH of the medium to or below a second pH value for a specified time period and subsequently restoring the pH to the first pH value.

In a preferred embodiment, the second pH value is selected from pH 3.5, 3.0, 2.5, 2.0, 1.5 and 1.0. In another preferred embodiment, the specified time period is selected from 2, 1.5, 1.0 and 0.5 hours. In a further preferred embodiment, the microalgae are selected from Nannochloropsis, Chlorococcum, and Nannochloris.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a flow diagram illustrating one embodiment of the method of the invention;

FIG. 2 is a schematic drawing illustrating an FGD process;

FIG. 3 is a schematic drawing illustrating one embodiment of a process to increase CO₂ concentration in the flue gas;

FIG. 4 is a schematic drawing illustrating the operation of a molecular sieve type carbon hollow fibre filter;

FIG. 5 is a sectional side view of the filter of FIG. 4 showing the movement of the various gases through the filter;

FIG. 6 is a graph illustrating CO₂ supply options to the algae farm as a function of distance and cost; and

FIG. 7 is a bar graph showing the average levels of the PUFAs arachidonic acid (AA), eicosapentaenoic (EPA), and docosahexaenoic (DHA), as a % of total fatty acids in the following microalgae: Chlorphyte (CHLOR), Prasinophyte (PRAS), Cryptophyte (CRYPT), Diatoms (DIAT), Rhodophyte (RHOD), Eustigmatophyte (EUST), Prymnesiophyte-Pavlova spp. (PYRM-1) and Prymnesiophyte-Isochrysis sp, (PYRM-2).

### DETAILED DESCRIPTION OF EXEMPLARY EMBODIMENTS

The method of the invention will be exemplified with reference to an installation built at the Ruthenberg Power Station (Ashkelon, Israel) of the Israel Electric Co. (IEC). However, it is to be emphasized that this is only an exemplary embodiment of the invention, and other embodiments will be obvious to the skilled man of the art.

### Overview of the Method

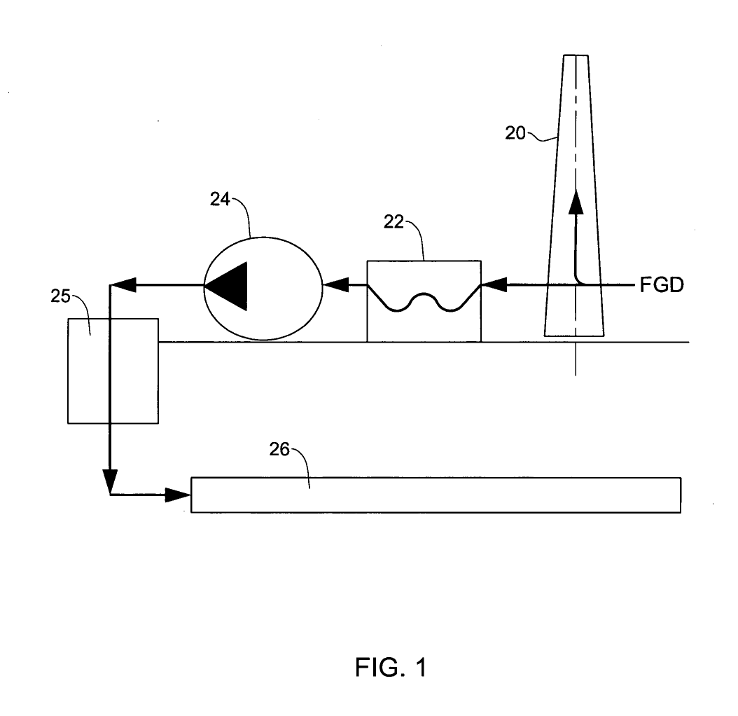

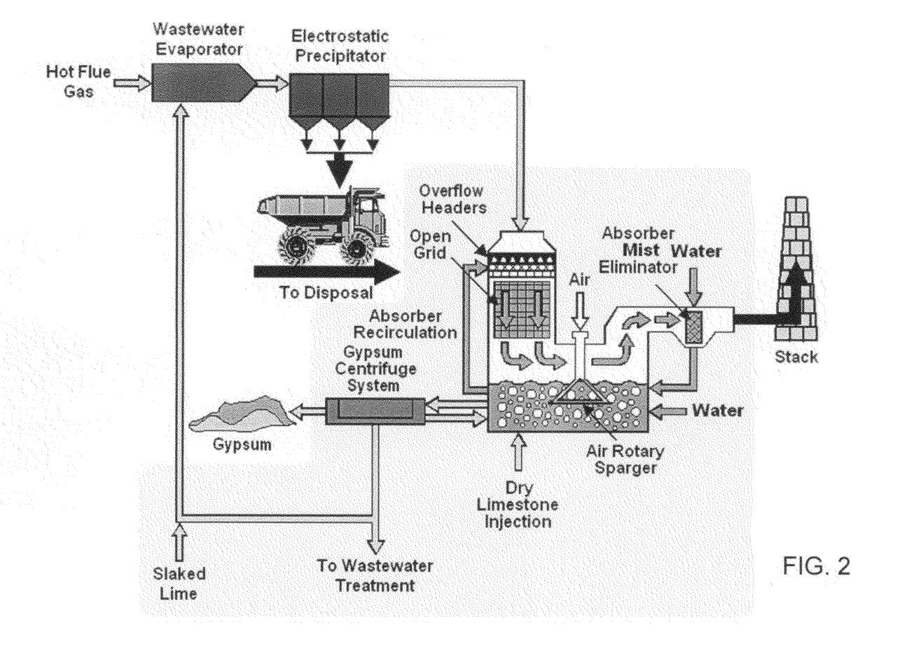

FIG. 1 provides a broad overview of the method of the invention. The flue gas produced by the (coal-based) power station generally undergoes FGD (wet scrubbing) before being released to the atmosphere through the smoke stack 20. In accordance with an embodiment of the method of the invention, the flue gas is shunted from the stack through a condensation tank 22, blower 24 and aftercooler 25 to the microalgae pond 26. An example of the FGD process is illustrated in FIG. 2. The FGD process (based on gypsum) reduces the SO₂ from ˜600 ppm to less than 60 ppm, i.e. by 90%.

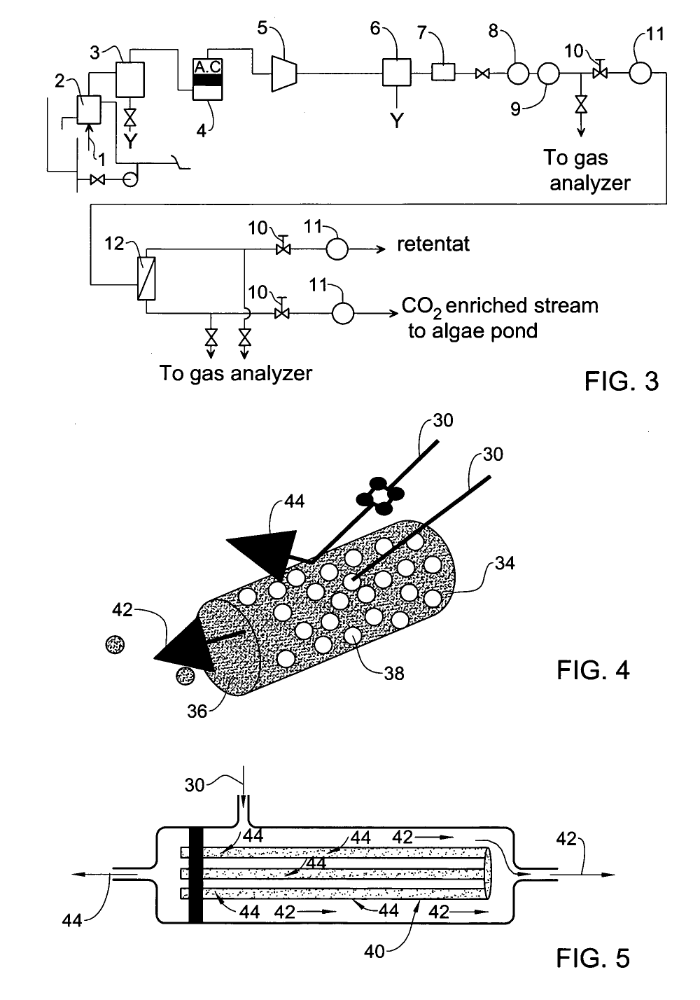

FIG. 3 shows a scheme of the experimental CO₂ concentrating system, mounted on the Rutenberg Power Station.

Flue gases (1) are cooled down in the cooler (2), pass the mist eliminator (3) and the filter (4) containing special activated carbon EcoSorb® granules, adsorbing NOₓ and SO₂. Afterwards, pressure is increased by the compressor (5), with the receiver tank (6) and the dried gas (7). Pressure (8 bar) is controlled by the pressure regulator (8) and measured by the manometer (9). Flow is controlled by the needle valve (10) and measured by the rotameter (11). Separation of gases is carried out by the carbon membrane (CMSM) (12). The pressure drop of flow gases at the carbon membrane is about 6 bar. The scrubbed, drained and concentrated flue gases are pumped through the pipeline by the compressor which is able to create an output pressure necessary to supply the gases to the microalgae pool.

### Separation Using Membranes

Membrane separation methods are particularly promising for CO₂ separation from low purity sources, such as the power plant flue gas, due to high CO₂ selectivity, achievable fluxes and favorable process economics. Porous membranes are microscopic sieves, which can separate molecules depending on molecular size or strength of interactions between molecules and the membrane surface. By a proper choice of the membrane pore size and surface properties, the transport of CO₂ across a membrane can be facilitated with respect to the transport of nitrogen and oxygen, leading to an efficient CO₂ separation process.

In accordance with one embodiment of the invention, the Carbon Molecular Sieve Membrane (CMSM), kindly provided by “Carbon Membranes Ltd” (CML) (Israel), was found to be suitable for use in the method of the invention. CML designs and manufactures gas separation systems based on unique hollow-fibre carbon molecular sieve technology.

As illustrated in FIGS. 4 and 5, molecular sieving is a mechanism whereby different molecules are separated based mainly on their different sizes. When a gas mixture 30 is fed into the shell 32 of a hollow fiber, it flows along the wall 34 of the fiber, attempting to permeate its wall and enter the bore 36. CMSM's uniqueness is in its ability to control the size of the pores 38 in the walls, to a resolution of tenths of Angstroms. Hence, when the pore size distribution is managed so that virtually all of the pore diameters fall between the size of the large and small molecules of the gas mixture, separation becomes possible. As the gas mixture is blown around the molecular sieve fiber 40, the molecules smaller than the pores 42 will readily penetrate through the fiber wall and will be concentrated in the fiber lumen. The larger molecules 44, on the other hand, cannot pass through the pores and hence will be concentrated on the outside of the fiber. This process can occur only with sufficient driving force, i.e. the partial pressure of the “faster” gas on the outer side of the membrane should at all times be higher than that on the inner side.

The separation module consists of a large number of fibers—typically 10,000—within a stainless steel shell. The module is carefully designed to ensure maximum circulation of the feed gas to optimize the separation process, along with durability to withstand field conditions.

The separation module is only as good as the system in which it operates. Potential configurations are multiple: typical systems can entail multiple modules working in parallel, in cascade, or both. Partial pressure differentials, being the key to the separation mechanism, are carefully controlled to optimize the system. Peripheral equipment is chosen to reach the best solution for the individual user, balancing costs with the technical performance of each option.

### EXAMPLE I

### Membrane Separation

One of the unique features of the CMSM manufacturing technology is the ability to strictly control the membrane permeability/selectivity combination in order to adjust it to various applications. In this regard, the membrane tested in this work was prepared to reach the optimum permeability/selectivity combination for air separation.

The results described below were obtained with a one-end-open type pilot module, composed of approximately 10,000 carbon hollow fibers, having an active separation area of 3.4 m².

The permeation measurements and air enrichment experiments were performed with single gases: N₂, O₂, CO₂ and SF₆. (The last gas was used in order to demonstrate the molecular sieving properties of the membrane). The experiments were carried out at room temperature and at a feed pressure of up to 5 bar.

Two sets of experiments were performed:
permeability measurements with pure gases;
air separation.

Considering that the carbon fibers are able to withstand pressures greater than 10 bar, the model was also used for predicting the separation process at higher applied pressure.

The results of the measurements of concentration of CO₂ and pollutants in flue gases of Ruthenberg Power Station IV unit scrubbed by FGD System carried out with and without use of the membrane CMSM are shown in Table 1.

TABLE 1
CO₂ and pollutants concentrations

| CO₂ | CO | SO₂ | NO | |
|---|---|---|---|---|
| in % | [ppm] | [ppm] | [ppm] | Note |
| 8.6-9.9% | 53-56 | 2 | 5-7 | After activated carbon filter |
| | | | | (without CMSM) |
| 29.6% | 56 | 3 | 4 | After activated carbon filter and |
| | | | | CMSM |

### EXAMPLE II

### Transport Systems

In one embodiment of a transport system for delivering the treated flue gases to the microalgae cultivation area, the following components are required:

1) a main gas pipeline adapted to transport a carbon dioxide-containing gas;

2) a primary gas manifold positioned proximate to a field of algae;

3) a trunk-line for delivering the carbon dioxide-containing gas from the main gas pipeline to the primary gas manifold; and

4) a plurality of secondary exhaust pipelines extending from the primary gas manifold into a pond and including exhaust ports for delivering a carbon dioxide-rich gas to the algae.

One of the major commercial considerations is the distance between the Power Unit which supplies the CO₂ and the Algae Farm. This distance dictates the option to be chosen. The larger amount of “parasitic” gases transferred, the more expensive pipes that have to be used, as well as more expenditure of energy due to gas compression.

On the other hand, pure CO₂ production involves the construction of a Mono-Ethanol-Amine (MEA) plant.

In the following calculation, the algae farm area is assumed to be 1000 ha. In order to provide efficient algae cultivation, 100 t/hr CO₂ shall be supplied.

The supply possibilities are:
Pure CO₂ after an MEA extraction process from the Power Unit stack.

The transportation is relatively cheap, because of the smaller pipe diameter, but the CO₂ separation plant is the main investment.
Flue Gas supply as is: 14.5% CO₂ after the FGD Plant and partial vapors condensation.
Enriched Flue Gas composition to 50% CO₂ by means of membrane separation.

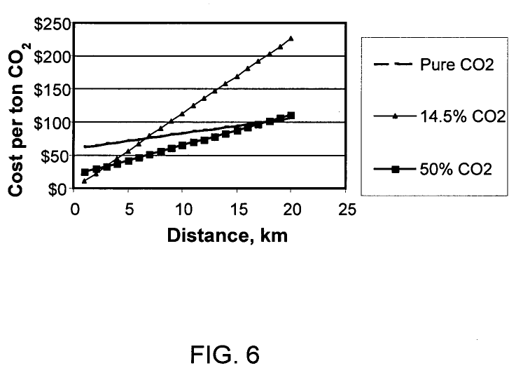

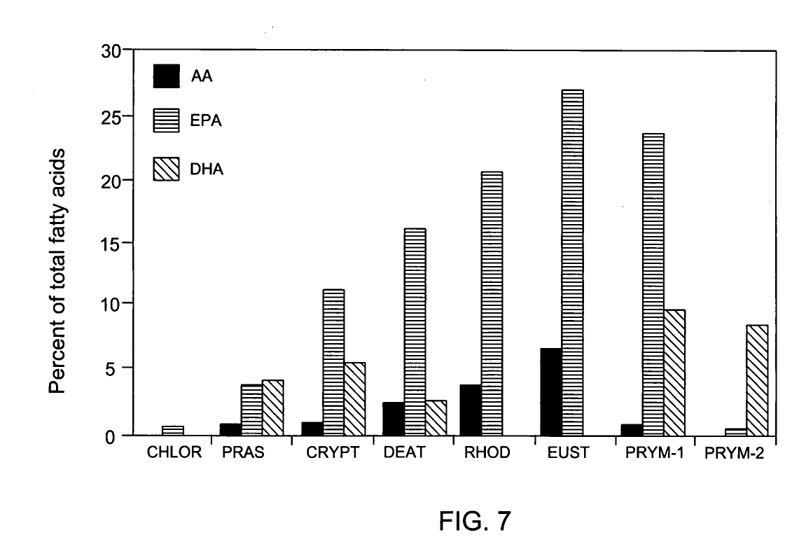

The aforementioned possibilities are summarized in FIG. 6, which indicates the ranges of costs of 1 ton of transported CO₂ due to the distance between the Power Station and the Algae Farm. The calculations are based on the data summarized in Table 2.

TABLE 2
Calculation of Pipeline System of Supply of Flue Gases and CO₂ to Seawater Ponds

| | | | 2. | 3. |
|---|---|---|---|---|
| | | 1. | Flue Gases | Flue Gases |
| | | Pure CO₂ | 14.5% CO₂ | 50% CO₂ |
| Technical | Mass Flow, kg/hr | 100,000 | 556,529 | 183,221 |
| Data | Pipeline diameter, m | 1 | 2 | 1.3 |
| | Compressor pressure, bars | 0.34 | 0.36 | 0.34 |
| | Compressor power consumption, kW | 669 | 5,963 | 1,869 |
| Financial | Total Investment, USD (millions) | 9,000,000 | 16,500,000 | 12,000,000 |
| Data | Investment (20 years loan @5%), per | 50,000 | 825,000 | 600,000 |
| | year | | | |
| | Investment per ton CO₂ | $1.29 | $2.36 | $1.71 |
| | Electricity price US $/kWh | 0.15 | 0.15 | 0.15 |
| | Electricity cost USD per ton CO₂ | 1.0 | 8.9 | 2.8 |
| | (assume 15 US cent/kWh) | | | |
| | Total transportation cost, $/ton CO₂ | $2.29 | $11.30 | $4.52 |
| | Separation cost, per ton CO₂ | 60 | 0 | 20 |
| | Total Sequestration cost | $62.29 | $11.30 | $24.52 |

Data in the table refers to 10 km distance.

It is very important to note, that by using flue gases with a high concentration of CO₂ (>90%), the level of concentration of harmful pollutants (as SO₂ and NOₓ) in seawater ponds will be much lower, than when non-enriched flue gases are used (<20% wt CO₂). Experience with the FGD system in the Ruthenberg Power Station has shown that content of SO₂ and other pollutants is much lower than design values, i.e. the values of the manufacturer's specifications (˜30 ppm instead of ˜200 ppm).

Exemplary results of measured gas volumes before and after FGD are given below.

TABLE 3
measured gas volumes before and after FGD

| Gas volume, | | |
|---|---|---|
| Nm³/kg fuel | Before FGD | After FGD |
| CO₂ (%) | 13.9 | 13.3 |
| SO₂ (ppm) | 500 | 56-70 |
| NOₓ (ppm) | 300 | 190-200 |

Exemplary results of metal concentrations before and after FGD are given below.

TABLE 4
metal concentrations (mg/dNm³) before and after FGD

| metal | Before FGD | After FGD |
|---|---|---|
| Ag | <0.01 | <0.01 |
| Al | 4.0 | 2.3 |
| As | <0.05 | <0.05 |
| B | 5.6 | 4.2 |
| Ba | 0.03 | 0.04 |
| Be | <0.01 | <0.01 |
| Ca | 4.1 | 2.3 |
| Cd | <0.005 | <0.005 |
| Co | <0.01 | <0.01 |
| Cr | 0.01 | <0.01 |
| Cu | <0.01 | <0.01 |
| Fe | 1.4 | 0.5 |
| Hg | <0.01 | <0.01 |
| K | 0.3 | 0.2 |
| Li | <0.01 | <0.01 |
| Mg | 0.9 | 0.6 |
| Mn | 0.03 | 0.01 |
| Mo | <0.01 | <0.01 |
| Na | 1.3 | 0.8 |
| Ni | <0.01 | <0.01 |
| P | 0.2 | 0.1 |
| Pb | <0.01 | <0.01 |
| S | 126 | 60 |
| Se | <0.01 | <0.01 |
| Sr | 0.1 | 0.06 |
| Ti | 0.1 | 0.05 |
| V | 0.01 | <0.01 |
| Zn | 0.03 | 0.02 |

The gas, after being treated by FGD, is then passed through a condensation tank, blower and aftercooler, prior to being introduced into the algae ponds. In one example, the component gas concentrations of this treated gas were measured.

TABLE 5
FGD gas impurities prior to being introduced into the algae ponds

| Gas | Concentration |
|---|---|
| CO₂ | 12.18-12.74% |
| NO | 173.7-185.7 ppmv |
| NO₂ | 22.8-23.1 ppmv |
| SO₂ | 29.0 ppmv |
| O₂ | 5.6% |
| CO | — |
| pH | 1-2 |

### EXAMPLE III

### Aeration

The supply of flue gases to ponds is carried out with the help of aeration equipment.

Aeration equipment is manufactured from chemically stable polymeric materials as aerated modules. A preferred example of aeration equipment is the KREAL tubular aerator (porous) (Russian Patent No. 32487). Aerated modules are made in the form of LPP (low pressure polyethylene) pipes in which the aerators are fixed in pairs by polyamide tees.

Aerating modules are carried out as LPP pipes (d=110-160 mm) on which aerators are fastened in pairs through a plastic trilling. Module breadth is 1.1 m; the step between aerators is 1.5-4 m. The change of a step between aerators allows changing ejection intensity over a wide range so that optimum CO₂ mode is assured.

The using of polymeric materials in aerated modules reduces the time of assembling and increases the term of the aerator's operation. KREAL porous aerators produce fine-bubble aeration (d=3 mm) in ponds. Their effectiveness at mass transfer of CO₂ from flue gases is 3 times higher than at aerators from perforated pipes.

TABLE 6
Technical characteristics of KREAL aerators

| | |
|---|---|
| Length, mm | 500 |
| Diameter external/internal, mm | 44/40 |
| Weight, kg | 0.2 |
| Pore diameter, micron | 40-100 |
| Working range of gas consumption, m³/hour | 2-10 |
| Head loss, mm of seawater column | 40 |
| Pressure loss of flue gases, mm of seawater column | 100 |
| Coefficient of aerators type by faz/fat = 0.2 | 1.8 |
| by faz/fat = 0.85 | 2.5 |

### EXAMPLE IV

### Algae

While growing algae in accordance with the method of the invention, it was unexpectedly found that two algae species grew at a rate significantly higher than usually found under standard cultivation conditions. These species were Skeletonema costatum and Nannochloropsis sp. The average productivity of Nannochloropsis and Skeletonema grown on coal burning flue gas after FGD was found to be approximately 20 g×m²×day⁻¹, as opposed to e.g. 4 g×m²×day⁻¹for Dunaliella grown on pure CO₂.

The growth conditions and characteristics for the period March 2005-November 2006 are summarized below:

Skeletonema costatum

### (Data at Bio-Max)

Algal Biomas, 0.5-1.5 g×L⁻¹

Cell number, no count

Chlorophyll a, 15 mg×L⁻¹; Carotenoids, 3-15 mg×L⁻¹

Car/chl, 0.3-1.0 (highly brown)

Turbine sea water at max; 450,000 m³/hr, 12-35° C.

Flue Gas after FGD at max, CO₂—431 t/hr, 10,344 tons CO₂/day;

Cultivation pH, 5-8 (IEC flue gas at pH 1)

Total dissolved carbon (TDC), 2-5 mM by IEC flue gas CO₂

N, P, by demand at optimum

Fe & minerals. Supply of essential minerals by the FGD gas.

### Nannochloropsis

### (Data at Bio-Max)

Algal Biomass, 0.5-1 g×L⁻¹

Cell number, 80-250×10⁹×L⁻¹

Chlorophyll a, 10-20 mg×L⁻¹; Carotenoids, 3-5 mg×L⁻¹

Car/chl, 0.3 (highly green, to avoid photo-inhibition)

Turbine sea water at max: 450,000 m³/hr, 12-35° C.

Flue Gas after FGD at max, CO₂—431 t/hr, 10,344 tons CO₂/day

pH of gas moisture, ˜1 (IEC flue gas)

Cultivation optimum pH ˜6.5

Requested TDC, 2-5 mM

N, P, by demand at optimum

Fe and minerals. Supply of essential minerals by the FGD gas

TABLE 7
Specifications and growth conditions of algae grown on coal burning flue gas and cooling turbine sea water.

| | | | | | | | | Average |
|---|---|---|---|---|---|---|---|---|
| | Growth | Growth | Algae | | | pH | Maximal | Algal |
| Marine Algal | Season | Temp. | Shape & | Sensitivity to | | Growth | Biomass Density | Productivity |
| Species | Israel | Range | size | contamination | Contamination | (0ptimum | In pond of 20 cm | (g/m2/day) |
| (Class) | (Months) | (min-max ° C.) | (μm) | (H, M, L) | Treatment* | range) | depth (g/L) | By Period |
| Chlorococcum | April-September | 18-35 | Sphere | L | Chlorine | 7.0-8.0 | 0.7 | ~20 |
| (Chlorophyceae) | | | 10 | | pH | | | |
| Dunaliella | All year | 10-32 | Oval | M | Detergents | 7.0-9.0 | 1.0 | ~20 |
| (Chlorophyceae) | | | 5 × 10 | | | | | |
| Nannochloris | May-October | 22-36 | Sphere 1 | M | Chlorine | 5.5-7.5 | 0.6 | ~20 |
| (Chlorophyceae) | | | | | pH | | | |
| Nannochloropsis | October-May | 5-25 | Sphere | M | Chlorine | 5.5-7.5 | 0.7 | ~20 |
| (Eustigmatophyceae) | | | 1.5 | | pH | | | |
| Skeletonema | May-October | 20-35 | Chain | M | | 7.0-8.0 | 1.0 | ~20 |
| (Bacillariophyceae) | | | | | | | | |
| Tetraselmis | April-September | 16-28 | Oval | L | Chlorine | 7.0-8.0 | 1.5 | ~20 |
| (Chlorophyceae) | | | 7 × 12 | | | | | |

Contamination treatment: chlorine, 1-3 ppm; Low pH.
Nutrients added to sea water: KNO₃, 0.1-5 mM; KH₂PO₄, 0.01-0.5 mM; FeCl₃, 0-30 μM

Many microalgae are sources of PUFA in general, and ω-3 fatty acids in particular, as can be seen in FIG. 6. Nannochloropsis (a member of EUST in FIG. 6) is known to be a source of ω-3 fatty acids (see for example U.S. Pat. No. 6,140,365, whose entire contents are incorporated herein), as is Skeletonema (a member of DIAT in FIG. 6). ω-3 fatty acids are known to be important for the human diet, and have various therapeutic and prophylactic effects, such as for treating cardiovascular, inflammatory, autoimmune and parasitic diseases.

An analysis of the fatty acid content of Nannochloropsis cultivated according to one embodiment of the method of the invention was carried out, and the results are presented in Table 8.

TABLE 8
Fatty Acids Analysis of Nannochloropsis

| Fatty Acid | % of Total Fatty Acids |
|---|---|
| Lauric (C12:0) | 0.5 |
| Myristic (C14:0) | 7.4 |
| Pentadecanoic (C15:0) | 0.4 |
| Palmitic (C16:0) | 22.6 |
| Palmitoleic (C16:1) | 28.5 |
| Heptadecanoic (C17:0) | 0.5 |
| cis-10-Heptadecenoic (17:1) | 0.6 |
| Stearic (C18:0) | 0.4 |
| Elaidic (C18:1n9t) | 3.2 |
| Oleic (C18:1n9c) | 0.4 |
| Linolelaidic (C18:2n6t) | 0.1 |
| Linoleic (C18:2n6e) | 2.6 |
| γ-Linolenic (C18:3n6) | 0.7 |
| not identified | 1.7 |
| Linolenic (C18:3n3) | 0.2 |
| cis-8,11,14-Eicosatrienoic (C20:3n6) | 0.3 |
| Arachidonic (C20:4n6) | 4.9 |
| cis-5,8,11,14,17-Eicosapentaenoic (C20:5n3) | 24.7 |

It may be seen that the Nannochloropsis contains an exceptionally high percentage of EPA (25% of total fatty acids, equivalent to 4% DW). Thus, the method of the invention can be used to prepare microalgae as a source for ω-3 fatty acids.

A similar analysis was carried out for Skeletonema cultivated according to the invention. The results are presented in Table 9.

TABLE 9
Fatty acid profile of Skeletonema

| Fatty Acid | % of Total Fatty Acids |
|---|---|
| Tridecanoic acid (C13:0) | 0.2 |
| Myristic acid (C14:0) | 1.3 |
| Myristoleic acid (C14:0) | 0.3 |
| Pentadecanoic acid (C15:0) | 0.2 |
| Palmitic (C16:0) | 25.8 |
| Palmitoleic acid (C16:0) | 7.3 |
| Heptadecanoic acid (C17:0) | 0.6 |
| cis-10-Heptadecenoic acid (C17:0) | 0.2 |
| Stearic (C18:0) | 2.1 |
| Oleic (C18:1n9c) | 30.3 |
| Linolelaidic (18:2n6e) | 5.2 |
| Linolenic (C18:3n3) | 12.6 |
| γ-Linolenic acid (C18:3n6) | 0.5 |
| Arachidonic (C20:4n6) | 4.1 |
| cis-11-Eicosenoic acid (C20:1) | 0.4 |
| 5,8,11,14,17-Eicosapentaenoic ((C20:5n3) | 5.7 |
| Arachidonic acid (C20:4n6) | 1.4 |
| Heneicosnoic acid (C21:0) | 0.4 |
| Docosahexanenoic acid (C22:6n3) | 1.4 |
| DHA-Docosahexaenoic acid (C22:6n3) | 0.054 |

In addition to ω-3 fatty acids, microalgae can be a source for biofuels such as biodiesal and bioethanol. The following results were obtained for the cellular lipid, protein and carbohydrate content (% of DW) of the six species cultivated according to the invention. The lipid content is important for biodiesal production, while the carbohydrate level is important for bioethanol production.

TABLE 10
Algal chemistry

| | | | | Pigments | | |
|---|---|---|---|---|---|---|
| | Chemical Composition | | | | | Carotenoids/ |
| Marine Algal Species | (% Ash free dry weight) | | | | Chl/cell | chlorophyll |
| (Class) | Lipids | Carbohydrate | Protein | Chlorophyll | (pg) | (g/g) |
| Chlorococcum | 15-25 | 30-50 | 25-55 | a + b | 5-12 | 0.21-0.25 |
| (Chlorophyceae) | | | | | | |
| Dunaliella | 15-25 | 30-60 | 15-50 | a + b | 0.5-5 | 0.25-10.0 |
| (Chlorophyceae) | | | | | | |
| Nannochloris | 10-30 | 25-55 | 20-50 | a + b | 0.01-0.03 | 0.20-0.26 |
| (Chlorophyceae) | | | | | | |
| Nannochloropsis | 7-30 | 15-40 | 20-60 | a | 0.05-0.12 | 0.18-0.24 |
| (Eustigmatophyceae) | | | | | | |
| Skeletonema | 15-35 | 15-45 | 20-40 | a + c | chains | 0.20-0.28 |
| (Bacillariophyceae) | | | | | | |
| Tetraselmis | 11-28 | 20-50 | 20-50 | a + b | 0.8-1.5 | 0.23-0.26 |
| (Chlorophyceae) | | | | | | |

Thus, it may be seen that the method of the invention can be used to prepare microalgae as a source for biofuels such as biodiesal and bioethanol.

While harvesting the Skeletonema, it was discovered that they promptly precipitate without centrifugation. This unexpected property of the algae grown in accordance with the method of the invention imparts a significant advantage to the harvesting of the algae, in that a centrifugation step of many cubic meters of culture is avoided. This presents a significant economic saving in the harvesting process.

While growing the algae, it was found that it was important to treat the seawater to prevent the growth of contaminants. Treatment was found to be important both before the addition of the algae as well as in the presence of the algae.

Thus, an additional aspect of the invention is a method of removing contaminants, and in particular protozoan contaminants, from an aqueous medium comprising microalgae, the medium having a first pH value, the method comprising lowering the pH of the medium to or below a second pH value for a specified time period and subsequently restoring the pH to the first pH value. In one embodiment, the second pH value is selected from pH 3.5, 3.0, 2.5, 2.0, 1.5 and 1.0. In another embodiment, the specified time period is selected from 2, 1.5, 1.0 and 0.5 hours. In a further embodiment, the microalgae are selected from Nannochloropsis, Chlorococcum, and Nannochloris.

The following is an exemplary treatment protocol of seawater in open ponds before adding the algae.

Stock solutions:

sodium hypochlorite 13%;

sodium thiosulfate 0.76 M

Procedure:
add 20 ppm sodium hypochlorite;
incubate at least 1 hour under continuous mixing;
add sodium thiosulfate at a 1:1 ratio to the sodium hypochlorite;
incubate at least 10 min. under continuous mixing;
check seawater chlorine concentration to verify neutralization.

The following is an exemplary treatment protocol for seawater in open ponds in the presence of Nannochloropsis algae.

Chlorine Treatment

Stock solution:

sodium hypochlorite 13%;

Procedure:
60-300 organisms—add 1 ppm sodium hypochlorite
300-600 organisms—add 2 ppm sodium hypochlorite
>600 organisms—add 3 ppm sodium hypochlorite
light and heat accelerate decomposition of sodium hypochlorite; therefore, it is not advisable to perform the treatment in daylight.
the lower the pH, the higher is the ratio of hypochlorous acid that has the disinfection effect; therefore, it is recommended to perform the treatment when pH is in the range of 5-6.

pH Treatment

Stock solution:

5M HCl; 5M NaOH

Procedure
add HCl to a final concentration of 2.5 mM, bringing the pH of the pond water to 2-3.5;
incubate for 1 hour;
add NaOH to a final concentration of 2.5 mM, thus restoring the original pH value.

The skilled man of the art will understand how to adapt the above protocol to other microorganisms and conditions by routine experimentation.

1. A method of growing photosynthetic organisms comprising providing said photosynthetic organisms with flue gases from a fossil-fuel power plant, the gases being treated by desulfurization.

2. The method of claim 1 wherein the carbon dioxide (CO₂) concentration of the flue gases is increased over the CO₂ concentration as released from the power plant.

3. A method of growing photosynthetic organisms comprising providing said photosynthetic organisms with flue gases from a fossil-fuel power plant wherein the CO₂ concentration of said flue gases is increased over the CO₂ concentration as released from the power plant.

4. The method of claim 1 wherein the fossil-fuel is selected from coal, petroleum, natural gas and biomass.

5. The method of claim 4 wherein the fossil-fuel is coal.

6. The method of claim 1 wherein the desulfurization is selected from wet scrubbing, spray dry scrubbing and dry sorbent injection.

7. The method of claim 2 wherein the CO₂ concentration is increased by a factor selected from 1.5, 2, 3, 4, 5 and 6.

8. The method of claim 2 wherein the CO₂ concentration is increased by a process using a low pressure preliminary condensation tank to remove water from the FGD treated gas flow.

9. The method of claim 2 wherein the CO₂ concentration is increased using a membrane unit.

10. The method of claim 9 wherein the membrane unit is a carbon molecular sieve type membrane.

11. The method of claim 10 wherein the carbon molecular sieve is a hollow fibre type.

12. The method of claim 9 wherein the CO₂ concentration is increased by a process using a tank (filter) with special activated carbon.

13. The method of claim 1 wherein the flue gases are passed through a filtering system for removing sulfur and/or nitrogen oxides.

14. The method of claim 10 wherein the CO₂ concentration is increased by a process using a compressor(s) station with one or more of control devices, valves, pipes, instruments and speed control facilities, as a part of the membrane unit.

15. The method of claim 2 wherein the CO₂ concentration is increased by a process using a gas receiver tank.

16. The method of claim 1 wherein the photosynthetic organisms are grown in a body of water, and the flue gases are dispersed in the body of water.

17. The method of claim 16 wherein the water is seawater.

18. The method of claim 16 wherein an aeration device is used for dispersion of the flue gas in the body of water.

19. The method of claim 18 wherein the aeration device is a porous aeration device.

20. The method of claim 16 wherein condensate (liquid) collected during the pretreatment of the flue gas is dispersed in the body of water in parallel with the flue gases.

21. The method of claim 1 wherein the photosynthetic organisms are microalgae.

22. The method of claim 21 wherein the microalgae are marine microalgae.

23. The method of claim 22 wherein the marine microalgae are selected from Bacillariophyta, Dinophyta, Chlorophyta, Cyanophyta and Eustigmatophyta.

24. The method of claim 23 wherein the marine microalgae are selected from Skeletonema, Nannochloropsis, Chlorococcum, Dunaliella, Nannochloris, and Tetraselmis.

25. A method for producing ω fatty acids comprising growing microalgae which are a source of ω fatty acids by providing said microalgae with flue gases from a fossil-fuel power plant.

26. The method of claim 25 further comprising separating the ω fatty acids from the microalgae.

27. A method for producing a biofuel comprising growing microalgae which are a source of biofuel by providing said microalgae with flue gases from a fossil-fuel power plant.

28. The method of claim 27 further comprising separating the biofuel from the microalgae.

29. The method of claim 27 wherein the biofuel is biodiesal or bioethanol.

30. A method of harvesting microalgae from a cultivation medium comprising growing the microalgae using flue gases from a fossil-fuel power plant, the gases being separated by desulfurization, allowing the microalgae to precipitate and harvesting the precipitated microalgae.

31. The method of claim 30 wherein the microalgae are Skeletonema.

32. A method of removing protozoan contaminants from an aqueous medium comprising microalgae, the medium having a first pH value, the method comprising lowering the pH of the medium to or below a second pH value for a specified time period and subsequently restoring the pH to the first pH value.

33. The method of claim 32 wherein the second pH value is selected from pH 3.5, 3.0, 2.5, 2.0, 1.5 and 1.0.

34. The method of claim 32 wherein the specified time period is selected from 2, 1.5, 1.0 and 0.5 hours.

35. The method of claim 32 wherein the microalgae are selected from Nannochloropsis, Chlorococcum, and Nannochloris.
